(19) United States
(12) Patent Application Publication
(10) Pub. No.: US 2023/0298728 A1
(43) Pub. Date: Sep. 21, 2023

(54) LINKING PATIENT-SPECIFIC MEDICAL DEVICES WITH PATIENT-SPECIFIC DATA, AND ASSOCIATED SYSTEMS AND METHODS

(71) Applicant: Carlsmed, Inc., Carlsbad, CA (US)

(72) Inventors: Niall Patrick Casey, Carlsbad, CA (US); Shariq Hussain, Vista, CA (US); Michael J. Cordonnier, Carlsbad, CA (US)

(21) Appl. No.: 18/108,422

(22) Filed: Feb. 10, 2023

Related U.S. Application Data
(63) Continuation of application No. PCT/US2021/045503, filed on Aug. 11, 2021.
Application No. PCT/US2021/045503 is a continuation-in-part of application No. 16/990,810, filed on Aug. 11, 2020.
(60) Provisional application No. 63/188,945, filed on May 14, 2021.

(51) Int. Cl. G16H 20/40; G16H 10/65; G16H 40/63

(52) U.S. Cl. CPC G16H 20/40; G16H 10/65; G16H 40/63

(57) ABSTRACT

Systems and methods for providing patient-specific medical devices and patient-specific surgical plans are described herein. In some embodiments, a patient-specific implant is provided that includes an implant body configured to interface with one or more identified anatomical structures at and/or proximate a target position. The patient-specific implant also includes a data storage element positioned on and/or within the implant body. The data storage element can include memory storing data that is accessible after the patient-specific implant is implanted in the patient. The data can include (i) first data specifying at least one step of a patient-specific surgical plan for implanting the patient-specific implant at the target position, and (ii) second data specifying one or more characteristics of the patient-specific implant.

### CROSS-REFERENCE TO RELATED APPLICATIONS

The present application is a continuation of International Patent Application No. PCT/US2021/045503, filed Aug. 11, 2021, which claims benefit under 35 U.S.C. § 119(e) of U.S. Provisional Patent Application No. 63/188,945, filed May 14, 2021 and is a continuation-in-part of U.S. Non-Provisional patent application Ser. No. 16/990,810, filed Aug. 11, 2020, the disclosures of which are incorporated by reference herein in their entireties.

### TECHNICAL FIELD

The present disclosure is generally related to designing and implementing medical care, and more particularly to systems and methods for designing and implementing patient-specific surgical procedures and/or medical devices.

### BACKGROUND

Orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis.

### BRIEF DESCRIPTION OF THE DRAWINGS

The accompanying drawings illustrate various embodiments of systems, methods, and devices, and various embodiments of other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Non-limiting and non-exhaustive descriptions are described with reference to the following drawings. The components in the figures are not necessarily to scale, emphasis instead being placed upon illustrating principles.

FIG. 1 is a network connection diagram illustrating a system for providing patient-specific medical care and configured in accordance with select embodiments of the present technology.

FIG. 2 illustrates a computing device suitable for use in connection with the system of FIG. 1, in accordance with select embodiments of the present technology.

FIG. 3 is a schematic illustration of a patient-specific implant contained within packaging and having a retrieval feature for accessing remotely stored patient-specific data, in accordance with embodiments of the present technology.

FIG. 4 is a schematic illustration of a patient-specific implant contained within packaging and having a data storage element for storing patient-specific data, in accordance with embodiments of the present technology.

FIG. 5 is a schematic illustration of a patient-specific implant having patient-specific markings in accordance with embodiments of the present technology.

FIG. 6 is a flowchart of a method for confirming the actual position of an implanted patient-specific implant is the same as a predetermined target position, in accordance with embodiments of the present technology.

FIG. 7 is a partially schematic illustration of an operative setup for accessing a patient-specific surgical plan and for implanting a patient-specific implant into a patient, in accordance with embodiments of the present technology.

FIGS. 8A-8C are partially schematic illustrations for obtaining patient-specific information from a patient-specific implant before, during, and after an operative procedure, in accordance with embodiments of the present technology.

### DETAILED DESCRIPTION

The present technology is directed to systems and methods for providing patient-specific medical devices, instruments, and surgical plans. For example, in many embodiments, the present technology provides a patient-specific implant that is designed to be implanted at a target position within a particular patient. In some embodiments, the present technology also includes patient-specific instruments and/or patient-specific surgical plans that can be used to deliver the implant. For example, the patient-specific surgical plan can include instructions for implanting the implant at the target position. To link the surgical plan to the implant, the implant and/or packaging associated with the implant can include at least one of a data storage element or a retrieval feature. The data storage element can include a memory storing the surgical plan and other patient-specific data. The retrieval feature can be associated with the surgical plan and other patient-specific data, and can be used to download or otherwise access a portion of or the entire surgical plan and other data, which can be stored on a remote server. In some embodiments, the implants may also include one or more markings, identifiers, and/or indicia (e.g., physical markings, digital markings, coded markings, etc.) in addition to or in lieu of the data storage element and retrieval features. The markings can be positioned on, within, or coupled to a body of the implant, and can include information about the patient, the implant, and/or the patient-specific surgical plan. Thus, the markings enable a user to directly identify information about the patient, the implant, and/or the surgical plan.

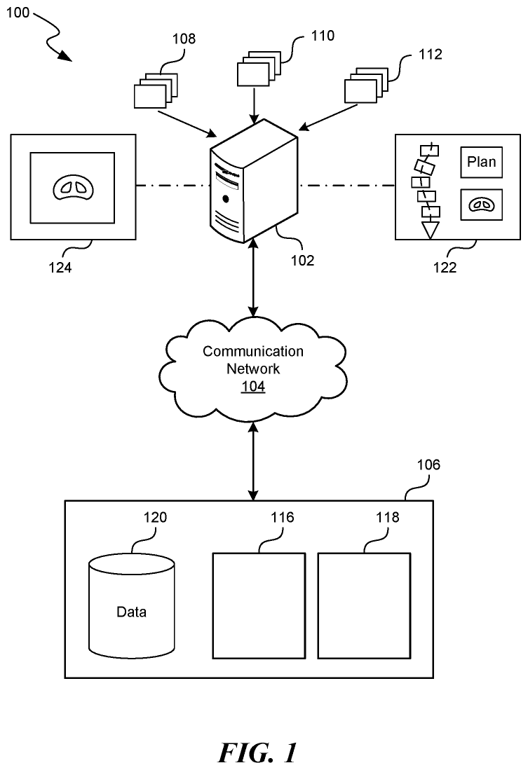

In some embodiments, the present technology includes systems that can design a patient-specific implant for implantation at a target location within a particular patient. A treatment planning module (e.g., treatment planning module 118 of FIG. 1) can analyze images of the patient to identify anatomical structures at or proximate to a target location suitable for engaging an implant. The anatomical structures can be selected based on one or more corrections, targeted anatomical structure spacing (e.g., intervertebral spacing between vertebral bodies), joint characteristics, target kinematic motion, or the like. The treatment planning module can use prior patient datasets to identify, select, and/or design features of an implant body. For example, the treatment planning module can identify design features from a group of candidate features, such as parametric features, dimensions, structural features, etc. The implant body can then be designed to provide a desired fit in the patient. A virtual model can be used to analyze the custom implant and to evaluate how structural features of the implant engage the identified anatomical structures. A designer can adjust the implant body design, eliminate structural features of the implant body, or add additional features to the implant design. The data associated with the implant design and design process, as well as the implant data, can be stored by the implant itself. If the implant is an artificial disc, for example, the stored data can include kinematic data (e.g., pre-operative patient data, target kinematic data, etc.), manufacturing data, design parameters, target service life data, physician recommendations/notes, etc. The disc can include an articulating implant body with plates contoured to match vertebral endplates, custom articulating members between the plates for providing patient-specific motion, etc. If the implant is an intervertebral cage, the stored data can include materials specifications of the implant body, dimensions of the implant body, manufacturing data, design parameters, target service life data, physician recommendations/notes, etc. After implantation, the physician can evaluate compression of the implant body by comparing the current dimensions of the implant body to original implant dimensions stored by the implant.

In some embodiments, the present technology is directed to providing patient-specific technology for a particular patient. The technology can include custom implants designed for delivery and implantation at a target site. The custom implant can be a patient-specific implant designed based on anatomical features at or adjacent to the implantation site. In some implementations, the implant can be designed to include structural features suitable for contacting identified anatomical features to improve treatment, reduce implant movement, etc. The structural features can be rigid surfaces (e.g., outer surfaces of an implant body), anchors, fixation features, etc. Images of the implantation site can be analyzed to identify the anatomical features. Because the implant has a custom configuration, a standard implantation procedure may be suboptimal. Accordingly, a patient-specific surgical plan can be generated to provide an optimal implantation procedure.

In some implementations, the implant can provide, or provide access to, the patient specific surgical plan to avoid human error, miscommunication of the plan, etc. For example, the implant can include a patient-specific marking that provides the implant information (e.g., lot number, material composition, manufacturing identification, etc.), patient information (e.g., patient name, birth date, etc.), treatment information (e.g., target implantation location for the implant, surgical access path, etc.), or the like. The marking can also indicate the implant has a data storage element and can provide information for accessing the stored information. Thus, in some embodiments, the implant includes a storage element with memory capable of storing the plan data retrievable by the physician, a robotic surgery system, or the like. In such embodiments, a robotic surgery system can retrieve the surgical plan and develop an appropriate patient-specific implantation procedure to simplify implant delivery. This avoids the risk of operator errors when planning the surgery or manually inputting data into the robotic surgery system. After implantation (e.g., months or years after surgery), a physician may want to obtain information about the customized implant not contained in the patient's medical records. The physician can use a reader or interrogator to non-invasively obtain implant data (e.g., implant dimensions, manufacture, ID, etc.) from the markings and/or storage element still within the patient. The storage element can be configured to permanently store the data while remaining in the patient's body. The physician can use the implant data when evaluating the patient and developing additional treatment plans. If the data in the storage element is incomplete or inaccurate, the data can be rewritten, modified, or erased.

In some embodiments, therefore, the present technology provides a patient-specific implant designed to be implanted at a target position within a particular patient. The patient-specific implant includes an implant body configured to interface with one or more identified anatomical structures at and/or proximate the target position. The patient-specific implant also includes a data storage element positioned on and/or within the implant body. The data storage element can include memory storing data that is accessible after the patient-specific implant is implanted in the patient. The data can include (i) first data specifying at least one step of a patient-specific surgical plan for implanting the patient-specific implant at the target position, and (ii) second data specifying one or more characteristics of the patient-specific implant.

In some embodiments, the present technology provides a patient-specific implant designed to be implanted at a target position within a particular patient. The patient-specific implant includes an implant body configured to interface with one or more identified anatomical structures at and/or proximate the target position. The patient-specific implant also includes a retrieval feature positioned on and/or within the implant body. The retrieval feature can be used to access (i) first data associated with a patient-specific surgical plan for implanting the patient-specific implant at the target position, and (ii) second data associated with the patient-specific implant. For example, the first data and the second data may be stored on a server, and the retrieval feature can be used to download or otherwise access the first data and the second data from the server.

In some embodiments, the present technology provides a patient-specific implant design to be implanted at a target position within a particular patient. The implant includes an implant body configured to interface with one or more identified anatomical structures at and/or proximate the target position. The implant also includes a patient-specific marking or identifier positioned on or within the implant body. The patient-specific marking or identifier can include or otherwise correspond to (i) first data associated with one or more elements of a patient-specific surgical plan for implanting the patient-specific implant at the target position, and (ii) second data associated with the patient-specific implant, and/or (iii) third data associated with the patient. For example, the first data, the second data, and/or the third data may be placed directly on the implant body, such that the patient-specific marking can be directly read from the implant body.

In some embodiments, the present technology provides a kit having a patient-specific implant contained within packaging. The packaging for the patient-specific implant can contain a data storage element and/or a retrieval feature. The data storage element contained within the packaging can include (i) first data associated with a patient-specific surgical plan for implanting the patient-specific implant at the target position, and (ii) second data associated with the patient-specific implant. The retrieval feature contained within or otherwise imprinted on the packing can be used to access (i) first data associated with a patient-specific surgical plan for implanting the patient-specific implant at the target position, and (ii) second data associated with the patient-specific implant. For example, the first data and the second data may be stored on a server, and the retrieval feature can be used to download or otherwise access the first data and the second data from the server.

In some embodiments, the present technology provides a system including a patient-specific implant designed to be implanted at a target position within a particular patient and a patient-specific surgical plan including instructions for implanting the patient-specific implant at the target position. The system can further include means for transmitting the patient-specific surgical plan to a surgical platform configured to execute one or more aspects of the surgical plan. For example, If the patient-specific surgical plan is stored locally, such as on a data storage element, the system can include a transmitter for transmitting the surgical plan to the surgical platform. In some embodiments, the transmitter is an antenna, one or more proximity sensors, or the like. If the patient-specific surgical plan is stored remotely, the system may include one or more retrieval features for downloading or otherwise accessing the remotely stored surgical plan. Once downloaded, the surgical plan can be transmitted to the surgical platform. In some embodiments, the surgical platform can be a robotic surgical platform configured to perform or otherwise assist with the one or more aspects of the surgical plan.

In some embodiments, the present technology provides methods for providing patient-specific medical care related to an implanted patient-specific implant. The method can include obtaining, from the implanted patient-specific implant, patient-specific data. The patient-specific data can include a predetermined optimal implant location for the patient-specific implant based on the patient's anatomy and/or condition. The method can further include obtaining an actual position of the patient-specific implant, such as by using one or more imaging techniques. The actual position can be compared to the predetermined optimal implant location to determine whether the actual position is the same as the predetermined optimal implant location, thus providing the patient with optimal benefit.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

The headings provided herein are for convenience only and do not interpret the scope or meaning of the claimed present technology.

### A. Systems for Designing Patient-Specific Implants and Patient-Specific Surgical Plans

FIG. 1 is a network connection diagram illustrating a computing system 100 for providing patient-specific medical care and configured in accordance with select embodiments of the present technology. As described in further detail herein, the system 100 is configured to design a patient-specific implant (also referred to herein as “implant,” unless the context clearly indicates otherwise) and/or generate a patient-specific surgical plan (also referred to herein as “surgical plan,” unless the context clearly indicates otherwise) for a patient. For example, the system 100 can generate an implant and/or generate a surgical plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof. The surgical plan can include surgical information, surgical plans (e.g., surgical implant procedure, target implant location and/or orientation, etc.), technology recommendations (e.g., device and/or instrument recommendations), and/or medical device designs. For example, the surgical plan can include at least one treatment procedure (e.g., a surgical procedure or intervention) and/or at least one medical device (e.g., an implanted medical device (also referred to herein as an “implant” or “implanted device”) or implant delivery instrument).

In some embodiments, the system 100 generates implants and/or surgical plans that are customized for a particular patient or group of patients, also referred to herein as a “patient-specific” or “personalized” implant and surgical plan. The patient-specific implant and/or patient-specific surgical plan can be designed and/or optimized for the patient's particular characteristics (e.g., condition, anatomy, pathology, condition, medical history). For example, the implant can be designed and manufactured specifically for the particular patient, rather than being an off-the-shelf device. However, it shall be appreciated that a patient-specific implant and/or a patient-specific surgical plan can also include aspects that are not customized for the particular patient. For example, a patient-specific surgical procedure can include one or more instructions, portions, steps, etc. that are non-patient-specific. Likewise, a patient-specific implant can include one or more components that are non-patient-specific, and/or can be used with an instrument or tool that is non-patient-specific. Personalized implant designs can be used to manufacture or select patient-specific technologies, including medical devices, instruments, and/or surgical kits. For example, a personalized surgical kit can include one or more patient-specific implants, patient-specific instruments, non-patient-specific technology (e.g., standard instruments, devices, etc.), instructions for use, patient-specific surgical plan information, or a combination thereof.

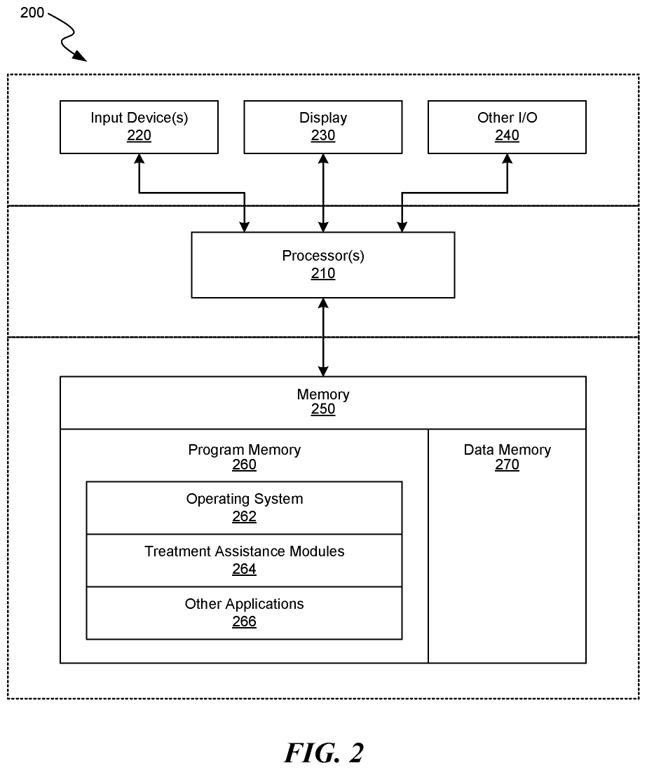

The system 100 includes a computing device 102, which can be a user device, such as a smart phone, mobile device, laptop, desktop, personal computer, tablet, phablet, or other such devices known in the art. As discussed in greater detail with reference to FIG. 2, the computing device 102 can include one or more processors, and memory storing instructions executable by the one or more processors to perform the methods described herein. The computing device 102 can be associated with a healthcare provider that is treating the patient. Although FIG. 1 illustrates a single computing device 102, in alternative embodiments, the computing device 102 can instead be implemented as a computing system encompassing a plurality of computing devices, such that the operations described herein with respect to the computing device 102 can instead be performed by the computing system and/or the plurality of computing devices.

The computing device 102 is configured to receive a patient data set 108 associated with a patient to be treated. The patient data set 108 can include data representative of the patient's condition, anatomy, pathology, symptoms, medical history, preferences, and/or any other information or parameters relevant to the patient. For example, the patient data set 108 can include surgical intervention data, treatment outcome data, progress data (e.g., physician notes), patient feedback (e.g., feedback acquired using quality of life questionnaires, surveys), clinical data, patient information (e.g., patient identification information, demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), future scheduled medical procedures (e.g., scheduled surgery date), or the like. In some embodiments, the patient data set 108 includes data representing one or more of patient identification information (e.g., number (ID), name, initials, etc.), age, gender, body mass index (BMI), lumbar lordosis, Cobb angle(s), pelvic incidence, disc height, segment flexibility, bone quality, rotational displacement, and/or treatment level of the spine.

In some embodiments, the computing device 102 can also be configured to receive a surgical team data set 110. The surgical team data set 110 can include data representative of the surgical team that will perform the surgery on the patient. For example, the surgical team data set 110 can include preferences of the surgical team (e.g., preferred implant techniques, preferred implant instruments/tools, etc.), experience of the surgical team (e.g., past procedures performed by the surgical team), scored outcomes of past procedures performed by the surgical team, or the like. As used herein, the term “surgical team” can refer to a group of healthcare practitioners that work together in an operating room during an implant procedure, or to one or more individual surgeons.

In some embodiments, the computing device 102 can also be configured to receive a facility or provider data set 112. The facility data set 112 can include data representative of the facility at which the patient's surgery will occur. For example, the facility data set 112 can include preferences of the facility (e.g., preferred implant techniques, preferred implant instruments/tools, etc.), experience of the facility (e.g., past procedures performed at the facility), scored outcomes of past procedures performed at the facility, infrastructure available to assist/perform the surgery (e.g., availability of robotic surgical platforms, as well as the type of “input” required to control the “output” of the robotic surgical platforms), or the like. As used herein, the term “facility” can refer to a single operating room facility, a hospital having multiple operating rooms, and/or a network of hospitals.

The computing device 102 is operably connected via a communication network 104 to a server 106, thus allowing for data transfer between the computing device 102 and the server 106. The communication network 104 may be a wired and/or a wireless network. The communication network 104, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 106, which may also be referred to as a “treatment assistance network” or “prescriptive analytics network,” can include one or more computing devices and/or systems. As discussed further herein, the server 106 can include one or more processors, and memory storing instructions executable by the one or more processors to perform the methods described herein. In some embodiments, the server 106 is implemented as a distributed “cloud” computing system or facility across any suitable combination of hardware and/or virtual computing resources.

The computing device 102 and server 106 can individually or collectively perform the various methods described herein for designing implants and/or generating surgical plans. For example, some or all of the steps of the methods described herein can be performed by the computing device 102 alone, the server 106 alone, or a combination of the computing device 102 and the server 106. Thus, although certain operations are described herein with respect to the server 106, it shall be appreciated that these operations can also be performed by the computing device 102, and vice-versa.

In some embodiments, the server 106 includes one or more modules for performing one or more steps of the treatment planning methods described herein. For example, in the depicted embodiment, the server 106 includes a data analysis module 116 and a treatment planning module 118. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

### i. Patient-Specific Implants

The computing device 102 and/or the server 106 can design a patient-specific implant (“implant”) based at least in part on the patient data set 108, the surgical team data set 110, and/or the facility data set 112. For example, the treatment planning module 118 can design, based off on any of the foregoing data inputs, the implant. In some embodiments, the implant includes a design for an orthopedic implant. Examples of such implants include, but are not limited to, screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.).

The implant can be designed to match the patient's existing anatomy and/or to provide a correction to the patient's existing anatomy. For example, as described in greater detail below, the treatment planning module 118 may analyze image data of the patient's native anatomy to determine whether an anatomical correction is needed. The image data may show the patient's native anatomical configuration (e.g., pre-operative anatomy), such as the geometry, orientation, and topography of various anatomical features. In some embodiments, for example, the image data may show (and/or be used to determine) various anatomical characteristics, including, but not limited to, vertebral spacing, vertebral orientation, vertebral translation, abnormal bony growth, abnormal joint growth, joint inflammation, joint degeneration, tissue degeneration, stenosis, scar tissue, lumbar lordosis, Cobb angle(s), pelvic incidence, disc height, segment flexibility, rotational displacement, and other spinal tissue characteristics. If an anatomical correction is not required, the treatment planning module 118 can design the implant to fit the patient's native anatomy. If an anatomical correction is required, the treatment planning module 118 can design the implant such that, when the implant is implanted in the patient, it provides the anatomical correction. Additional details for designing patient-specific implants to provide one or more desired anatomical corrections can be found in U.S. application Ser. No. 16/987,113, filed Aug. 6, 2020, the disclosure of which is incorporated by reference herein in its entirety.

In some embodiments, the generated implant design is a design for an entire device. Alternatively, the generated design can be for one or more components of a device, rather than the entire device. In some embodiments, the implant design is for one or more patient-specific device components that can be used with standard, off-the-shelf components. For example, in a spinal surgery, a pedicle screw kit can include both standard components and patient-specific customized components. In some embodiments, the generated design is for a patient-specific medical device that can be used with a standard, off-the-shelf delivery instrument. For example, the implants (e.g., screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The implants described herein are expected to improve delivery into the patient's body, placement at the treatment site, and/or interaction with the patient's anatomy.

### ii. Patient-Specific Surgical Plans

The computing device 102 and/or the server 106 can also design a patient-specific surgical plan (“surgical plan”) based on the patient data set 108, the surgical team data set 110, and/or the facility data set 112. The surgical plan can include a detailed procedure for implanting the implant to a specific target position within the patient. For example, the surgical plan can include aspects of a pre-operative plan (e.g., detection and measurement of patient's anatomy, preparation of patient for a surgical procedure, etc.), a surgical procedure, a surgical approach (e.g., implant technique), one or more surgical steps (preparing tissue for an incision, making an incision, making a resection, removing tissue, manipulating tissue, performing a corrective maneuver, delivering the implant to a target site, deploying the implant at the target site, adjusting the implant at the target site, manipulating the implant once it is implanted, securing the implant at the target site, explanting the implant, suturing tissue, etc.) a target position, site, or location of the implant (e.g., a location, orientation, etc.), and other aspects related to pre-operative, operative, or post-operative plans.

In some embodiments, the surgical plan includes an orthopedic surgical procedure such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). Spinal surgery can also include non-fusion surgeries, such as artificial disc replacements. In some embodiments, the surgical procedure includes descriptions of and/or instructions for performing one or more aspects of a patient-specific surgical procedure. For example, the surgical procedure can include one or more of a surgical approach, a corrective maneuver, or a bony resection.

In some embodiments, the surgical plan includes a target position of the implant. As used herein, the terms “target position,” “target site,” and “target location,” refers to a predetermined optimal location for the implant to be placed during the implant procedure, and can be based on the patient's anatomy, condition, diagnosis, prognosis, activity-level, and the like. For example, the target position may be defined by one or more of the following parameters taken in relation to an anatomical landmark: an angle or degree of orientation, and angle or degree of translation, an insertion depth, an insertion angle, degree of contact between two surfaces, and the like. Suitable anatomical landmarks include, for example, specific vertebrae, specific vertebral levels, or other recognizable anatomical features. The target position can therefore include a three-dimensional position of the implant relative to patient anatomy (e.g., as defined by boundaries created by patient anatomy), and/or a target orientation of the implant relative to patient anatomy. In some embodiments, the target position may incorporate a desired correction to the patient's native anatomy such that, when the implant is implanted at the target position, it manipulates the patient's anatomy to achieve the desired correction. Without being bound by theory, placing the implant at the target position is expected to optimize the benefit of and/or minimize the side effects of the implant. In particular, the full benefit of the implant may only be realized when the implant is accurately placed at the target position.

In some embodiments, the surgical plan optionally includes a recommendation to remove tissue to clear space for the implant at the target position. For example, the surgical plan may include instructions to perform an osteotomy, muscular resection, soft tissue detachment, soft tissue retraction, or the like to prepare the patient to receive the patient-specific implant. In some embodiments, the surgical plan includes a manipulation of tissue to prepare the patient to receive the implant. For example, the surgical plan may include instructions to adjust a relative position of two vertebrae, increase a distance between two vertebrae, or the like.

In some embodiments, the surgical plan includes machine-readable instructions for carrying out various steps of the surgical plan. The machine-readable instructions can be configured such that, when executed by a surgical robotic platform, the machine-readable instructions cause the surgical robotic platform to execute various aspects of an operative procedure associated with implanting the implant. For example, the surgical platform may prepare tissue for an incision, make an incision, make a resection, remove tissue, manipulate tissue, perform a corrective maneuver, deliver the implant to a target site, deploy the implant at the target site; adjust a configuration of the implant at the target site, manipulate the implant once it is implanted, secure the implant at the target site, explant the implant, suture tissue, and the like. The instructions may therefore include particular instructions for articulating robotic arms, instruments, and/or tools to perform or otherwise aid in the delivery of the patient-specific implant.

In some embodiments, the surgical plan includes step-by-step written, verbal, and/or graphic instructions that show a surgeon how to perform the patient-specific surgical plan. The patient-specific surgical plan can be displayed to the surgeon before and/or during the operative procedure (e.g., via display 122). In some embodiments, the written, verbal, and/or graphic instructions can be encoded in computer-readable instructions. The encoded instructions can be decoded and displayed to the surgeon before and/or during the operative procedure. In some embodiments, the patient-specific surgical plan includes both machine-readable instructions and written, verbal, and/or graphic illustrations.

### iii. Using Reference Patient Data Sets to Design Patient-Specific Implants and/or Patient-Specific Surgical Plans

In some embodiments, the system 100 may consider one or more reference data sets when designing the patient-specific implant and/or the patient-specific surgical plan. For example, in some embodiments the server 106 includes at least one database 120 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 120 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, preferences, and/or any other information or parameters relevant to the reference patient, such as any of the data described herein with respect to the patient data set 108. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine. As another example, a reference patient data set can include treatment data regarding at least one treatment procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, activity level, return to work, complications, recovery times, efficacy, mortality, and/or follow-up surgeries.

In some embodiments, the server 106 receives at least some of the reference patient data sets from a plurality of healthcare provider computing systems. Each healthcare provider computing system can include at least one reference patient data set (e.g., reference patient data sets) associated with reference patients treated by the corresponding healthcare provider. The reference patient data sets can include, for example, kinematic records, electronic medical records, electronic health records, biomedical data sets, etc.

In embodiments in which the implant and/or the surgical plan is designed based on the reference data, the data analysis module 116 can include one or more algorithms for identifying a subset of reference data from the database 120 that is likely to be useful in developing a treatment plan. For example, the data analysis module 116 can compare patient-specific data (e.g., the patient data set 108 received from the computing device 102) to the reference data from the database 120 (e.g., the reference patient data sets) to identify similar data (e.g., one or more similar patient data sets in the reference patient data sets). The comparison can be based on one or more parameters, such as age, gender, BMI, pathology, kinematics, lumbar lordosis, pelvic incidence, and/or treatment levels. The parameter(s) can be used to calculate a similarity score for each reference patient. The similarity score can represent a statistical correlation between the patient data set 108 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients. In some embodiments, the data analysis module 116 includes one or more algorithms that select a set or subset of the reference patient data based on criteria other than patient parameters, such as the surgical team data set 110 (e.g., based on surgeon expertise, outcomes of particular types of procedures performed by the surgeon, etc.) and/or the facility data set 112 (e.g., surgical equipment such as surgical robots).

The data analysis module 116 can further be configured with one or more algorithms to select a subset of the reference patient data sets, e.g., based on similarity to the patient data set 108 and/or treatment outcome of the corresponding reference patient. For example, the data analysis module 116 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, range of motion, kinematic data, HRQL, activity level, complications, recovery times, efficacy, mortality, or follow-up surgeries. As described in further detail below, in some embodiments, the data analysis module 116 calculates an outcome score by assigning values to each outcome parameter. A patient can be considered to have a favorable outcome if the outcome score is above, below, or at a specified threshold value.

In some embodiments, the data analysis module 116 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 116 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific treatment plan to perform a battlefield surgery can be based on reference patient data from similar battlefield surgeries and/or datasets associated with battlefield surgeries. In another example, the patient-specific treatment plan can be generated based on available robotic surgical systems. The reference patient data sets can be selected based on patients that have been operated on using comparable robotic surgical systems under similar conditions (e.g., size and capabilities of surgical teams, hospital resources, etc.).

In embodiments in which the implant and/or the surgical plan is designed based on the reference data, the treatment planning module 118 can include one or more algorithms that generate the implant and/or the surgical plan based on the reference data. In some embodiments, the treatment planning module 118 is configured to develop and/or implement at least one predictive model for generating the treatment plan, also known as a “prescriptive model.” The predictive model(s) can be developed using clinical knowledge, statistics, machine learning, AI, neural networks, or the like. In some embodiments, the output from the data analysis module 116 is analyzed (e.g., using statistics, machine learning, neural networks, AI, etc.) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a treatment plan will produce a favorable outcome for the particular patient. The predictive model(s) can be validated, e.g., by inputting data into the model(s) and comparing the output of the model to the expected output.

In some embodiments, the treatment planning module 118 is configured to generate the implant design based on previous treatment data from reference patients. For example, the treatment planning module 118 can receive a selected subset of reference patient data sets and/or similar patient data sets from the data analysis module 116, and determine or identify treatment data from the selected subset. The treatment data can include, for example, range of motion and/or other kinematic data, treatment procedure data (e.g., surgical procedure or intervention data) and/or medical device design data (e.g., implant design data) that are associated with favorable or desired treatment outcomes for the corresponding patient. The treatment planning module 118 can analyze the treatment procedure data and/or medical device design data to determine an optimal treatment protocol for the patient to be treated. For example, the treatment procedures and/or medical device designs can be assigned values and aggregated to produce a treatment score. The patient-specific treatment plan can be determined by selecting treatment plan(s) based on the score (e.g., higher or highest score; lower or lowest score; score that is above, below, or at a specified threshold value). The personalized treatment plan can be based on, at least in part, the patient-specific technologies or patient-specific selected technology.

Alternatively or in combination, the treatment planning module 118 can generate the implant designs based on correlations between data sets. For example, the treatment planning module 118 can correlate implant designs and medical device design data from implant designs for similar patients with favorable outcomes (e.g., as identified by the data analysis module 116). Correlation analysis can include transforming correlation coefficient values to values or scores. The values/scores can be aggregated, filtered, or otherwise analyzed to determine one or more statistical significances. These correlations can be used to determine treatment procedure(s) and/or medical device design(s) that are optimal or likely to produce a favorable outcome for the patient to be treated.

Alternatively or in combination, the treatment planning module 118 can generate designs using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 118 generates the treatment plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 120, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training dataset can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate the treatment plan, the patient data set 108, the surgical team data set 110, and/or the facility data set 112 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one treatment plan for the patient. In embodiments where multiple trained machine learning models are used, the models can be run sequentially or concurrently to compare outcomes and can be periodically updated using training data sets. The treatment planning module 118 can use one or more of the machine learning models based the model's predicted accuracy score.

The implant design and/or the surgical plan generated by the treatment planning module 118 can be transmitted via the communication network 104 to the computing device 102 for output to a user (e.g., clinician, surgeon, healthcare provider, patient). For example, the communication network 104 can transmit the surgical plan to the computing device 102 in response to receiving a unique code associated with the implant and/or surgical plan, as described in greater detail below. In some embodiments, the computing device 102 includes or is operably coupled to a display 122. The display 122 can show various aspects of a surgical procedure to be performed on the patient, such as the surgical approach, treatment levels, corrective maneuvers, tissue resection, and/or implant placement. To facilitate visualization, a virtual model of the surgical procedure can be displayed. Additionally or alternatively, the display 122 can show a design for the implant, such as a two- or three-dimensional model of the device design. The display 122 can also show patient information, such as two- or three-dimensional images or models of the patient's anatomy where the surgical procedure is to be performed and/or where the device is to be implanted. The display 122 can also display structural features of the implant suitable for contacting anatomical features to improve treatment, reduce implant movement, etc. The structural features can be rigid surfaces (e.g., outer surfaces of an implant body), anchors, fixation features, etc. Images of the implantation site can be analyzed to identify such anatomical features identified by the treatment planning module 118. The computing device 102 can further include one or more user input devices (not shown) allowing the user to modify, select, approve, and/or reject the displayed treatment plan(s).

### iv. Manufacturing Patient-Specific Implants

In some embodiments, the patient-specific implant design generated by the treatment planning module 118 can be transmitted from the computing device 102 and/or the server 106 to a manufacturing system 124 for manufacturing a corresponding medical device. The manufacturing system 124 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. For example, the manufacturing system 124 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 124 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 124 can manufacture one or more patient-specific medical devices based on fabrication instructions or data (e.g., CAD data, 3D data, digital blueprints, stereolithography data, or other data suitable for the various manufacturing technologies described herein). In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

### v. Additional Features

The treatment plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 118 generates control instructions configured to cause a surgical robot (e.g., robotic surgery systems, navigation systems, etc.) to partially or fully perform a surgical procedure. The control instructions can be transmitted to the robotic apparatus by the computing device 102 and/or the server 106.

Following the treatment of the patient in accordance with the treatment plan, treatment progress can be monitored over one or more time periods to update the data analysis module 116 and/or treatment planning module 118. Post-treatment data can be added to the reference data stored in the database 120. The post-treatment data can be used to train machine learning models for developing patient-specific treatment plans, patient-specific medical devices, or combinations thereof.

It shall be appreciated that the components of the system 100 can be configured in many different ways. For example, in alternative embodiments, the database 120, the data analysis module 116 and/or the treatment planning module 118 can be components of the computing device 102, rather than the server 106. As another example, the database 120, the data analysis module 116, and/or the treatment planning module 118 can be located across a plurality of different servers, computing systems, or other types of cloud-computing resources, rather than at a single server 106 or computing device 102.

Additionally, in some embodiments, the system 100 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like. In some embodiments, the system 100 may include additional features and/or capabilities, such as any of those described in U.S. application Ser. No. 16/735,222, filed Jan. 6, 2020, U.S. application Ser. No. 17/342,439, filed Jun. 8, 2021, U.S. application Ser. No. 16/207,116, filed Dec. 1, 2018, and U.S. application Ser. No. 16/990,801, filed Aug. 11, 2020, the disclosures of which are incorporated by reference herein in their entireties.

FIG. 2 illustrates a computing device 200 suitable for use in connection with the system 100 of FIG. 1, according to an embodiment. The computing device 200 can be incorporated in various components of the system 100 of FIG. 1, such as the computing device 102 or the server 106. The computing device 200 includes one or more processors 210 (e.g., CPU(s), GPU(s), HPU(s), etc.). The processor(s) 210 can be a single processing unit or multiple processing units in a device or distributed across multiple devices. The processor(s) 210 can be coupled to other hardware devices, for example, with the use of a bus, such as a PCI bus or SCSI bus. The processor(s) 210 can be configured to execute one more computer-readable program instructions, such as program instructions to carry out of any of the methods described herein.

The computing device 200 can include one or more input devices 220 that provide input to the processor(s) 210, e.g., to notify it of actions from a user of the computing device 200. The actions can be mediated by a hardware controller that interprets the signals received from the input device and communicates the information to the processor(s) 210 using a communication protocol. Input device(s) 220 can include, for example, a mouse, a keyboard, a touchscreen, an infrared sensor, a touchpad, a wearable input device, a camera- or image-based input device, a microphone, or other user input devices.

The computing device 200 can include a display 230 used to display various types of output, such as text, models, virtual procedures, surgical plans, implants, graphics, and/or images (e.g., images with voxels indicating radiodensity units or Hounsfield units representing the density of the tissue at a location). In some embodiments, the display 230 provides graphical and textual visual feedback to a user. The processor(s) 210 can communicate with the display 230 via a hardware controller for devices. In some embodiments, the display 230 includes the input device(s) 220 as part of the display 230, such as when the input device(s) 220 include a touchscreen or is equipped with an eye direction monitoring system. In alternative embodiments, the display 230 is separate from the input device(s) 220. Examples of display devices include an LCD display screen, an LED display screen, a projected, holographic, or augmented reality display (e.g., a heads-up display device or a head-mounted device), and so on.

Optionally, other I/O devices 240 can also be coupled to the processor(s) 210, such as a network card, video card, audio card, USB, firewire or other external device, camera, printer, speakers, CD-ROM drive, DVD drive, disk drive, or Blu-Ray device. Other I/O devices 240 can also include input ports for information from directly connected medical equipment such as imaging apparatuses, including MRI machines, X-Ray machines, CT machines, etc. Other I/O devices 240 can further include input ports for receiving data from these types of machine from other sources, such as across a network or from previously captured data, for example, stored in a database.

In some embodiments, the computing device 200 also includes a communication device (not shown) capable of communicating wirelessly or wire-based with a network node. The communication device can communicate with another device or a server through a network using, for example, TCP/IP protocols. The computing device 200 can utilize the communication device to distribute operations across multiple network devices, including imaging equipment, manufacturing equipment, etc.

The computing device 200 can include memory 250, which can be in a single device or distributed across multiple devices. Memory 250 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 250 is a non-transitory computer-readable storage medium that stores, for example, programs, software, data, or the like. In some embodiments, memory 250 can include program memory 260 that stores programs and software, such as an operating system 262, one or more treatment assistance modules 264, and other application programs 266. The treatment assistance module(s) 264 can include one or more modules configured to perform the various methods described herein (e.g., the data analysis module 116 and/or treatment planning module 118 described with respect to FIG. 1). Memory 250 can also include data memory 270 that can include, e.g., reference data, configuration data, settings, user options or preferences, etc., which can be provided to the program memory 260 or any other element of the computing device 200.

### B. Linking Patient-Specific Implants and Patient-Specific Data Sets

As provided above, the present technology includes systems, devices, and methods for designing patient-specific technology, devices (e.g., implants, instruments, etc.), diagnostic plans, and surgical plans. In many embodiments, the surgical plans are specifically tailored to the implants (i.e., the surgical plans provide instructions for implanting the implant at a target position). Thus, the present technology further provides systems, devices, and methods for associating or otherwise linking a surgical plan with a corresponding implant to ensure the appropriate surgical plan can be accessed/used for implanting the corresponding implant. In addition to the surgical plan, the present technology can further link patient-specific implant information and other patient information to the implant (the patient-specific surgical plan, the patient-specific implant information, and the other patient information are collectively referred to as “patient-specific data”, “patient-specific data set”, or “data set”). Accordingly, aspects of the present technology ensure the patient-specific data set is stored with the corresponding implant and/or is readily accessible in combination with the corresponding implant.

The patient-specific data set can include a surgical plan, implant information, and/or other patient information. As previously described, the surgical plan can include aspects of pre-operative plans (e.g., detection and measurement of patient's anatomy, preparation of patient for a surgical procedure, etc.), a surgical procedure, a surgical approach (e.g., implant technique), one or more surgical steps (preparing tissue for an incision, making an incision, making a resection, removing tissue, manipulating tissue, performing a corrective maneuver, delivering the implant to a target site, deploying the implant at the target site; adjusting the implant at the target site; manipulating the implant once it is implanted, securing the implant at the target site, explanting the implant, suturing tissue, etc.) a target position of the implant (e.g., a location, orientation, etc.), and other aspects related to pre-operative, operative, or post-operative plans. The implant information can include implant dimensions, implant composition, unique implant identification code, implant part number, implant lot code, implant manufacture date, implant implantation date, implant expiration date, implant sterilization history, and/or implant manufacturing history. The other patient information can include data representative of the patient's condition, anatomy, pathology, symptoms, medical history, preferences, and/or any other information or parameters relevant to the patient. For example, the other patient information can include surgical intervention data, treatment outcome data, progress data (e.g., physician notes), patient feedback (e.g., feedback acquired using quality of life questionnaires, surveys), clinical data (e.g., lumbar lordosis, Cobb angle(s), pelvic incidence, disc height, segment flexibility, bone quality, rotational displacement, and/or treatment level of the spine), patient information (e.g., patient ID number, demographics, sex, age, gender, BMI, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like.

In some embodiments, the patient-specific data set is stored remotely (e.g., apart from the implant and implant packaging, such as in the “cloud” and/or on a server (e.g., the server 106 shown in FIG. 1)). In such embodiments, the implant or associated packaging can include one or more retrieval features for downloading and/or otherwise accessing the data set. In some embodiments, the patient-specific data set is stored locally (e.g., on, within, or adjacent the implant and/or the implant packaging). In such embodiments, the implant or associated packaging can include one or more data storage elements for locally storing the data set. In some embodiments, the data set is stored both locally and remotely. In some embodiments, some aspects of the data set are stored locally, and some aspects of the data set are stored remotely.

### i. Remote Storage of Patient-Specific Data Set

As provided above, in some embodiments the patient-specific data set is stored remotely, such as on the server 106. In such embodiments, the server 106 may store multiple patient-specific data sets for multiple different patients. Each individual patient-specific data set can be associated with a unique identifier, such as a unique code (e.g., alpha-numeric codes, including randomly generated alpha-numeric codes, person identification numbers (PIN), patient medical record numbers (MRN), patient names, or the like). Individual patient-specific data sets can be downloaded from the server 106 using the unique identifiers. For example, a user can send a request to the server 106 (e.g., using computing device 102 and communication network 104) to download a particular patient-specific data set by including the unique identifier with the request. The server 106 can compare the unique identifier received from the user with the unique identifiers associated with the stored patient-specific data sets. If the unique identifier received from the user matches a unique identifier associated with a stored patient-specific data set, the server 106 can retrieve the stored patient-specific data set and transmit it to the computing device 102 for display to the user. If the unique identifier received from the user does not match a unique identifier associated with a stored patient-specific data set, the server 106 can transmit a message or other alert to the computing device 102 for display to the user that indicates no patient-specific data set with the unique identifier was found on the server 106. Although the foregoing describes querying the server 106 to retrieve a particular patient-specific data set, the same or similar technique can be used to identify and retrieve a particular patient-specific data set from the cloud.

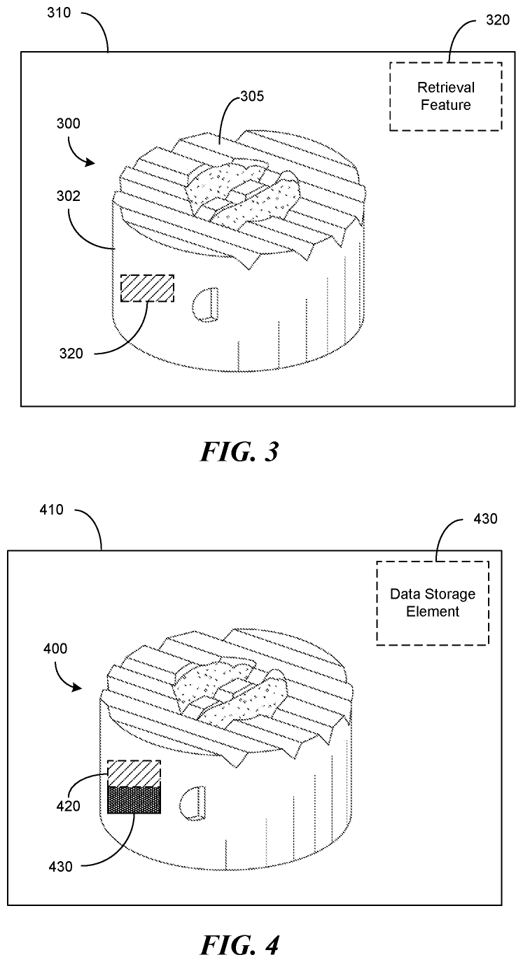

FIG. 3 is a schematic illustration of a patient-specific implant 300 contained within packaging 310 and associated with a remotely stored patient-specific data set. Although the implant 300 is shown as an intervertebral implant, one skilled in the art will understand that the implant 300 is merely one example of a patient-specific implant, and that the present technology is neither limited to intervertebral implants of the type shown, nor to intervertebral implants in general. The implant 300 has a body 302 configured to interface with one or more identified anatomical structures (e.g., one or more vertebral bodies or endplates) at and/or proximate the target implantation site (e.g., between one or more vertebral bodies or endplates). The implant body 302 includes one or more structural features designed to engage one or more identified anatomical structures. For example, in the illustrated embodiment, the implant 300 can include an upper surface 305 and a lower surface (not shown) configured to seat against vertebral bodies. In some embodiments, the upper surface 305 and the lower surface can have contours that match contours of the vertebral endplates, such that the upper surface 305 and lower surface “mate” with the corresponding vertebral endplates they engage with. In some embodiments, such as the illustrated embodiment, the upper surface 305 and/or the lower surface can be textured (e.g., via roughenings, knurlings, ridges, and the like). For lordotic correction, the upper surface 305 and the lower surface may be angled with respect to one another. In other procedures, the implant 300 can be an interspinous spacer with structural features in the form of U-shaped portions designed to receive respective spinous processes. The dimensions of the U-shaped portions can match the dimensions of the spinous processes. In other embodiments, the structural features can include recesses, arms, or other contact features designed to engage (e.g., contact, receive, etc.) one or more anatomical features (e.g., tissue, bony structures, etc.). Additional implant types, designs, and structural features suitable for engaging identified anatomical features are described, for example, in U.S. application Ser. No. 16/207,116, filed Dec. 1, 2018, and U.S. application Ser. No. 16/987,113, filed Aug. 6, 2020, the disclosures of which are incorporated by reference herein in their entireties.

As illustrated, the implant 300 and/or the packaging 310 can include one or more retrieval features 320 for accessing the remotely-stored patient-specific data set. The retrieval features 320 can be machine-readable (MR) graphics, a quick-response (QR) code, a bar code, or another feature associated with the unique identifier. In other embodiments, the retrieval feature 320 can include the unique identifier itself (e.g., the unique identifier can be an alpha-numeric code directly printed on the implant 300, printed on the packaging 310 for the implant 300, printed on an insert included in the implant packaging 310, or combinations thereof).

A user (e.g., a physician) can access the patient-specific data set using the retrieval feature 320. For example, in embodiments in which the retrieval feature 320 is a bar code corresponding to the unique identifier, the user can scan the retrieval feature 320 using, for example, one or more cameras on the computing device 102 and/or otherwise input the unique identifier into the computing device 102. Once the unique identifier is inputted into the computing device 102, the computing device 102 can send the unique identifier to the server 106 (e.g., via communication network 104) with a request to provide the corresponding patient-specific surgical data set. In response to the request, and as described above, the server 106 can locate the specific data set associated with the unique identifier and transmit the data set to the computing device 102 for display to the user. Although FIG. 3 illustrates that both the implant 300 and the packaging 310 include a retrieval feature 320, in other embodiments, only one of the implant 300 or the packaging contains the retrieval feature 320.

Once downloaded, the patient-specific data set can be used pre-operatively to review the surgical plan, during the operation to guide a surgeon and/or a robotic surgical platform to perform various steps of the surgical plan, and post-operatively to confirm correct placement of the implant 300. As previously described, the surgical plan can include a detailed patient-specific procedure for implanting the implant 300 to a specific target position within the patient. In some embodiments, the surgical plan includes machine-readable instructions for carrying out various steps of the patient-specific surgical plan. The machine-readable instructions can be configured such that, when executed by a surgical robotic platform, the machine-readable instructions cause the surgical robotic platform to execute various aspects of an operative procedure associated with implanting the implant 300. For example, the surgical platform may prepare tissue for an incision, make an incision, make a resection, remove tissue, manipulate tissue, perform a corrective maneuver, deliver the implant to a target site, deploy the implant at the target site, adjust the implant at the target site, manipulate the implant once it is implanted, secure the implant at the target site, explant the implant, suture tissue, and the like.

### ii. Local Storage of Patient-Specific Data Set

As provided above, in some embodiments the patient-specific data set is stored locally, such as on or within the patient-specific implant and/or on or within the packaging for the implant. FIG. 4 is a schematic illustration of a patient-specific implant 400 contained within packaging 410 having one or more data storage elements 430 with memory that stores the patient-specific data set. Although two data storage elements 430 are illustrated, in some embodiments a single data storage element 430 is provided, and is either positioned within or on the implant 400, or is contained within the packaging 410. Moreover, although the implant 400 is shown as an intervertebral implant, one skilled in the art will understand that the implant 400 is merely one example of a patient-specific implant, and that the present technology is neither limited to intervertebral implants of the type shown, nor to intervertebral implants in general. For example, the implant 400 can include any of the designs, structures, and the like described above with respect to the implant 300.

The data storage element 430 can be any suitable data storage medium, including but not limited to memory chips (e.g., microchips) having non-volatile memories such programmable ROM, erasable programmable ROM, electrically erasable programmable ROM, flash memory, mask ROM, and the like. In some embodiments, the data storage element 430 is a radio-frequency identification (RFID) chip. The RFID chip can be active (e.g., powered by a battery or other energy storage component) or passive (e.g., powered by energy from the RFID reader's energy). The data storage element 430 can be integral with the implant 400, can be physically coupled to the implant 400, or can otherwise be associated with the implant 400 (e.g., included in the packaging 410 with the implant 400). In embodiments in which the data storage element 430 is physically coupled to the implant 400, the data storage element 430 may optionally be releasably coupled to the implant 400 such that it can be removed from the implant 400 and connected to a computing device to access the patient-specific data set stored thereon. In other embodiments, the data storage element 430 is coupled to the patient-specific implant and is configured to be implanted with the implant 400. In some embodiments, the data storage element 430 is biocompatible (e.g., includes a biocompatible coating, is composed of biocompatible materials, etc.).

In embodiments in which the data storage element 430 is contained within or otherwise coupled to the implant 400, the implant 400 can optionally include one or more transmitters or retrieval features 420 for establishing a wireless connection with the data storage element 430. The one or more transmitters or retrieval features 420 can include, but are not limited to MR codes, QR codes, bar codes, and/or telecommunication features (e.g., antennas, proximity sensors, location sensors, radio-frequency transmission features, wireless transmission features, etc.). The wireless connection can be established using WiFi, Bluetooth, RF communication, Near-Field-Communication, or other suitable wireless communication techniques. To establish a wireless connection with the data storage element 430, a computing device can utilize the retrieval feature 420. For example, in embodiments in which the retrieval feature 420 is a bar code, the user can scan the retrieval feature 420 using one or more cameras on the computing device 102 (shown in FIG. 1). Once the retrieval feature 420 is scanned, the computing device 102 can automatically establish a wireless connection with the data storage element 430. In embodiments in which the retrieval feature 420 is a proximity sensor, the data storage element 430 may automatically establish a wireless connection with the data storage element 430 when a computing device or other scanner is brought within a specific distance of the data storage element 430. In embodiments in which the retrieval feature 420 is an antenna, a wireless connection can be established between a computing device and the data storage element 430 by directing RF energy towards the antenna or otherwise exposing the retrieval feature 420 to a magnetic and/or electric field. As one skilled in the art will appreciate, the necessity of having a retrieval feature 420 will depend on, for example, the type of wireless connection formed with the data storage element 430, and therefore in some embodiments the retrieval feature 420 will be omitted.

Once a wireless connection is established with the data storage element 430, the data storage element 430 may automatically transmit some or all of the patient-specific data to an external device (e.g., computing device 102). In other embodiments, once the wireless connection is established, a user can request some or all of the patient-specific data be retrieved from the data storage element 430, and, in response to the request, the data storage element can transmit the requested patient-specific data set. Once the patient-specific data set is retrieved from the data storage element 430, the data set can be used pre-operatively to review the surgical plan, during the operation to guide a surgeon and/or a robotic surgical platform to perform various steps of the surgical plan, and post-operatively to confirm correct placement of the implant 400, as previously described. Additional details of retrieving information from the data storage element 430 after the implant 400 has been implanted are described below in section (B)(iii).

In some embodiments, the data storage element 430 can be paired with other data storage elements in a peer-to-peer relationship to form a Blockchain of data. For example, in some embodiments, the patient may include more than one implant 400, and each implant 400 may include a corresponding data storage element 430. Each data storage element can contain, in addition to a first patient-specific data set associated with the implant it is connected to, a second patient-specific data set associated with the other implant(s) that it is not physically coupled to. Additionally, the data storage element 430 can contain data corresponding to the relationship between the multiple implants, such as whether they were implanted at the same time, implanted by the same surgeon, implanted at the same facility, or the like. Accordingly, a user can access either or both of the first patient-specific data set and the second patient-specific data set by communicating with a single implant. Without being bound by theory, this is expected to simplify the process of retrieving data sets when the patient has multiple implants. Moreover, distributing the data sets over a plurality of data storage elements enables the data storage elements 430 to be encrypted as part of a decentralized network that is associated with (and travels with) the patient.

In embodiments in which the data storage element 430 is included in the packaging 410 or is removably coupled to the implant 400, the patient-specific data set can be accessed by connecting the data storage element 430 to a computing device. Connecting can be either physically connecting, such as plugging in or inserting the data storage element 430 into the computing device, or wirelessly connecting, such as using WiFi, Bluetooth, RF communication, Near-Field-Communication, or other suitable techniques. In either case, connecting the data storage element 430 to the computing device can enable a user to access the data set stored on the data storage element 430.

### iii. Patient-Specific Markings Printed Directly on Implant

In addition to or in lieu of the retrieval features 320 shown in FIG. 3 and/or the data storage element 430 shown in FIG. 4, the devices described herein can include one or more patient-specific markings or identifiers (e.g., symbols, words, and/or letters) printed directly on the implant itself and that are associated with (e.g., includes some of) the information included within the patient-specific data set. FIG. 5, for example, illustrates an implant 500 configured in accordance with embodiments of the present technology. The implant 500 can be generally similar to the implants 300 and 400 described with respect to FIGS. 3 and 4 (with or without the retrieval features and data storage element). However, relative to the implants 300 and 400, the implant 500 includes one or more patient-specific markings 525 printed directly on a surface 502 of the implant 500. The patient-specific markings 525 can include some or all of the data included within the patient-specific data set. For example, the patient-specific markings 525 can include a textual representation of patient name, patient initials, a date (e.g., a patient intake date, a surgery date, an implant manufacturing date, etc.), a target location in the spine of the patient at which the implant is intended for insertion (e.g., L4-L5 disc space), a treatment level (e.g., a vertebral level) of the spine of the patient, data about the implant 500 (e.g., a size, a dimension, a shape, a material, an implant ID, a manufacturer, etc.). The patient-specific markings 525 can be printed, typed, written, engraved, or otherwise imprinted on the implant 500 in any language and using any suitable technique for adding text to an object. In some embodiments, the patient-specific markings 525 can be radio-opaque for viewing via fluoroscopy or other suitable methods.

In addition to or in lieu of the textual information, the patient-specific markings 525 can include machine-readable (MR) features (e.g., images, symbols, graphics, etc.), optically machine-readable features, one or more codes (e.g., a QR code, a bar code, and alpha-numeric code, etc.), identifier(s) associated with the patient-specific data set, and/or any combination thereof. However, even in embodiments in which the patient-specific markings 525 include encrypted information in the form of, for example, one or more codes, the patient-specific markings 525 still directly encode a portion of the patient-specific data set (e.g., the patient's name). This enables a user to access the portion of the patient-specific data set without needing to access a remote server. This is also in contrast with the retrieval feature 320 described with respect to FIG. 3, which enables the user to access the patient-specific data by providing access to the remotely-stored patient-specific data set.

A user (e.g., a clinician, a physician, etc.), computing device, and/or imaging system can use the one or more patient-specific markings 525 to obtain information associated with the patient and/or implant directly from the implant itself. For example, the user can read the information (e.g., the patient's name, the surgery date, the target implant level, etc.) directly form the implant 500, rather than having to retrieve/access information stored locally (e.g., on a data storage element 430) or remotely (e.g., on the server 10). However, in some embodiments, a user may also utilize the information provided by the patient-specific markings 525 (e.g., the patient's name or MRN) to access additional data from the patient-specific data set stored remotely, similar to the process described above with respect to FIG. 3 for using the retrieval feature 320 to access a data set stored remotely. In some embodiments, the patient-specific marking can be configured to be readable before, during, and/or after implantation, such as by an X-ray, a CAT scan, an MRI, and/or by a radiological measurement.

In some embodiments, the patient-specific markings 525 are included on the implant 500 in combination with the retrieval feature 320 and/or the data storage element 430. In such embodiments, the patient-specific markings 525 can be used to verify the information obtained using the retrieval feature 320 and/or the data storage element 430. For example, a user can use the patient specific markings 525 to determine one or more elements of the patient-specific data set (e.g., the patient's name and the intended vertebral level) by directly imaging, reading, or observing the implant. The user can then compare these elements with the same type of information (e.g., the patient's name and the intended vertebral level) obtained using the retrieval feature 320 and/or from the data storage element 430. In this way, the user can confirm that the information obtained using the retrieval feature 320 and/or the data storage element 430 is indeed associated with the implant 500.

The patient-specific markings 525 can be formed using any suitable process or technique. For example, the patient-specific markings 525 can be formed using an embossing or a debossing process, extruded from a surface of the implant 500 (e.g., via additive manufacturing), printed on the implant 500, or etched into the surface of the implant 500 (e.g., by selectively removing material from the implant 500). In some embodiments, the patient-specific markings 525 are configured to be permanently or releasably coupled to the implant 500. For example, the patient-specific markings 525 can be formed on a substrate, and the substrate can be coupled to the implant 500 by a fastener, an adhesive, and/or any other suitable coupling process or technique.

Without being bound by theory, use of the patient-specific markings 525 enables a surgeon or other user to quickly obtain and/or verify information about the patient and/or implant by simply reading the information printed on the implant. This is expected to be a quicker process than accessing remotely stored patient data (e.g., using the retrieval feature 320 shown in FIG. 3) and/or locally stored patient data on a computer-readable data storage element (e.g., the data storage element 430 shown in FIG. 4). However, the amount of information that can be provided using patient-specific markings 525 is of course limited by the size and shape of the implant 500 itself. Moreover, depending on the implant target and positioning of the implant, the patient-specific markings 525 may be difficult to locate and/or read once the implant 500 has been implanted in the patient. Accordingly, in some embodiments and as described above, implants in accordance with the present technology include patient-specific markings 525 in addition to a retrieval feature (e.g., the retrieval feature 320 or 420) and/or a data storage element (e.g., the data storage element 430), described with respect to FIGS. 3 and 4, respectively.

### iv. Accessing the Patient-Specific Data Set After Implantation

In some embodiments, the patient-specific data set can be accessed after the implant is implanted in the patient (e.g., days, months, or years after surgery). For example, in embodiments in which the implant includes a data storage element (e.g., as shown in FIG. 4), the data storage element is implanted with the implant. After the implant is implanted, the data set can still be retrieved from the data storage element using various wireless communication techniques. For example, in some embodiments, an external device can retrieve information from the data storage element after implantation using RF communication.

In some embodiments, the implant includes an antenna or other conducting element (e.g., the transmitter/retrieval feature 420 on patient-specific implant 400) for establishing RF communication with an external device (e.g., computing device 102, shown in FIG. 1). In some embodiments, one or more portions of the implant can be designed to act as an antenna for the data storage element. For example, in some embodiments the implant is an interbody device, and the lattice of the interbody device is designed to act as an antenna for the data storage element. A lattice or other three-dimensional structure of the implant can function as a non-directional antenna capable of transmitting data when a directional or non-directional energy field is present. Without being bound by theory, using the lattice or other three-dimensional structure as the transmitting or conducting element enables the data storage element to respond to RF energy without having to first orient the data storage element (and thus the patient) in a specific direction. In some embodiments, the implant has multiple directional or non-directional lattice or other three-dimensional structures each capable of transmitting when a respective field is applied. In some embodiments, the implant includes a dedicated structure that acts as an antenna for communicating with the external device. Regardless of the design, the antenna is configured to receive information from the external device and/or is configured to transmit information stored in the data storage element to the external device. For example, in some embodiments, the data storage element receives, via the antenna, a request for some or all of the patient-specific data set. In response to the request, the data storage element transmits, via the antenna, some or all of the data set to the external device. In some embodiments, rather than transmitting the data set itself, the data storage element can transmit a unique identifier to the external device. The unique identifier can be used to access the data set stored remotely, such as on server 106. The unique identifier can be the same as the unique identifiers previously described.

In some embodiments, accessing the patient-specific data set after implanting the implant 400 can be used to verify various aspects of the implant and/or the implant procedure. For example, in some embodiments, the data storage element 430 (or the retrieval feature 320 or the patient-specific markings 525) may include a unique identifier that is specific to the patient, pathology, implant procedure, section of anatomy, or the like. At any time after implantation, the unique identifier can be retrieved from the data storage element 430 and compared to a database storing information about the particular patient (e.g., database 120 shown in FIG. 1). As previously described, the database 120 can store medical records for the patient, which may include a description of the surgical procedure the patient underwent to receive the patient-specific implant. The medical records may also include the unique identifier associated with the patient-specific implant. The unique identifier retrieved from the data storage element 430 can thus be compared to the unique identifier stored with the patient's medical records to verify the implant 400 as authentic. In some embodiments, data can be transmitted back to the implant 400, indicating that the implant 400 was verified as authentic.

Accessing the patient-specific data set after implanting the implant 400 is expected to be useful for a number of additional reasons. For example, as previously discussed, the data set can include information about the implant, including, but not limited to, implant dimensions, implant composition, unique implant identification code, implant part number, implant lot code, batch number implant manufacture date, implant implantation date, implant expiration date, implant sterilization history, and/or implant manufacturing history. A medical professional or other user may therefore wish to access the data set after the implant is implanted to review and/or verify information about the implant, such as, for example, that the implant was properly sterilized before being implanted into the patient. The data set can further include information about the patient, such as medical history, diagnosis, prognosis, and the like. A medical profession or other user may therefore wish to access the data set after the implant is implanted to review medical history, confirm whether the surgical outcome was consistent with the patient's prognosis, or the like. In some embodiments, a medical professional can use the data set when evaluating the patient and developing additional treatment plans. The data set may further include the target position of the implant. A medical professional or other user may therefore wish to access the data set to confirm that the actual position of the implant is consistent with the target position, as described below.

### C. Confirming Positioning of the Patient-Specific Implant

The patient-specific implant may also include one or more positioning features that enable a user to post-operatively confirm the positioning of the implant. As previously described, the patient-specific surgical plan can include identifying a target position for the implant, which defines the optimal location for the implant based on the patient's condition, the patient's native anatomy, a desired correction to the patient's native anatomy, or the like. The one or more positioning features enable a user to confirm that the implant is located at the target position.

In some embodiments, the one or more positioning features can include the data storage element (e.g., the data storage element 430 on the patient specific implant 400). For example, in addition to storing and transmitting information as described above, the data storage element can be configured to transmit a position of the implant. In particular, the data storage element can transmit a positioning signal (e.g., an RF positioning signal) indicative of a location of the data storage element. The data storage element can generate and transmit the signal itself, and/or can reflect a signal transmitted from an energy source positioned external to the body. The transmitted position can be compared to an anticipated position of the data storage element associated with the target position of the implant. If the transmitted position is the same as (or within a threshold degree of deviation from) the anticipated position, the implant can be confirmed as positioned at the target position. If the transmitted position is not the same as (or is not within a threshold degree of deviation from) the anticipated position, the implant is not at the target position.

In some embodiments, the one or more positioning features can include one or more sensors. The one or more sensors can be configured to detect, measure, or otherwise determine a position of the implant. The detected position can be transmitted to an external device (e.g., computing device 102, shown in FIG. 1) and compared to the target position. If the detected position is the same as (or within a threshold degree of deviation from) the target position, the implant can be confirmed as positioned at the target position. If the detected position is not the same as (or is not within a threshold degree of deviation from) the target position, the implant is not at the target position.

In some embodiments, an image of the implanted implant can be constructed using conventional imaging techniques (e.g., X-Ray, RF imaging techniques, such as return strength signal, MRI, CAT-scan, etc.). The constructed image can be compared to a virtual model showing the implant implanted in the patient at the target position to determine whether the patient-specific implant is at the target position. In some embodiments, an image analysis module, such as an artificial intelligence architecture, can be used to compare the constructed image to the virtual model. In some embodiments, the image analysis module is a convolutional neural network. In some embodiments, the one or more positioning features can include one or more radiographic markers to aid in comparing the constructed image to the virtual model.

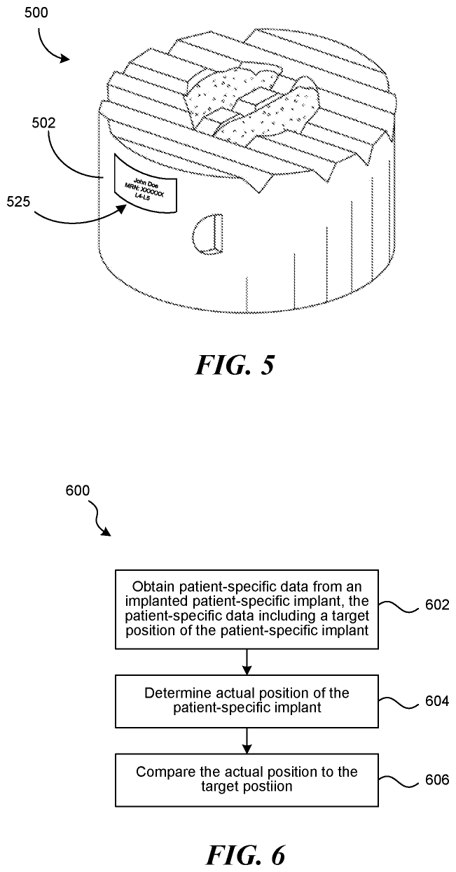

FIG. 6 is a flowchart of a method 600 for confirming the position of an implanted patient-specific implant. The method 600 can include obtaining, from the implant, patient-specific data in step 602. The patient-specific data can include a target position for the implant that defines a predetermined optimal location for the implant based on the patient's anatomy and/or condition. The patient-specific data can be obtained from the implant using any of the techniques previously described herein. For example, in some embodiments, the implant includes a data storage element storing the patient-specific data. In such embodiments, obtaining the patient-specific data in step 602 can include receiving the patient-specific data from the data storage element. The patient-specific data can be obtained from the data storage element as previously described, such as by exposing the implant to an electric and/or magnetic field, and/or exposing the implant to RF energy. In some embodiments, the implant includes a retrieval feature that encodes a unique identifier. In such embodiments, obtaining the patient-specific data in step 602 can include using the retrieval feature to obtain the unique identifier, and using the unique identifier to access the patient-specific data stored remotely, such as on a server.

In addition to the target position, the patient-specific data can include other patient-specific information, such as any of the information previously described herein. For example, the patient-specific data can include patient data and implant data. The patient data can include patient information, patient medical history, a patient medical record number, physician notes about the patient, and the like. The implant data can include implant dimensions, implant composition, unique implant identification code, implant part number, implant lot code, implant manufacture date, implant implantation date, implant expiration date, implant sterilization history, implant manufacturing history, and the like. The patient-specific data may further include other aspects related to the surgical plan used to implant the implant.

The method 600 can further include determining an actual position of the implant in step 604. In some embodiments, the actual position of the implant can be determined by capturing image data of the implanted implant. Suitable image data can include, for example camera images, Magnetic Resonance Imaging (MRI) images, ultrasound images, Computerized Aided Tomography (CAT) scan images, Positron Emission Tomography (PET) images, X-Ray images, and the like. Additionally or alternatively, the actual position of the patient-specific implant can be determined using one or more positioning elements included on the implant, as previously described herein.

The method 600 can further include, in step 606, comparing the actual position of the implant obtained in step 604 to the target position of the implant obtained in step 602. The comparison can be done manually, semi-automatically, or fully automatically. For example, in some embodiments, comparing the actual position to the target position includes using the image analysis module, as previously described. If the actual position is not the same as the target position (or within a threshold degree of deviation from the target position), the method can optionally provide an alert identifying that the actual position is not the same as the target position and/or provide one or more recommended corrective procedures for moving the implant from the actual position to the target position. If the actual position is the same as the target position (or within a threshold degree of deviation from the target position), the method can optionally provide an alert confirming that the actual position is aligned with the target position.

The foregoing techniques can be used to confirm proper placement of the implant during and/or after the implant procedure. For example, in some embodiments, any of the foregoing techniques can be used to confirm the implant is positioned at the target position before ending the implant surgery. The foregoing techniques can also be used at post-operative follow-up patient visits to ensure that the implant remains at the target position (e.g., confirm that it has not shifted or been ejected).

### D. Patient-Specific Implants and Robotic Surgery Platforms

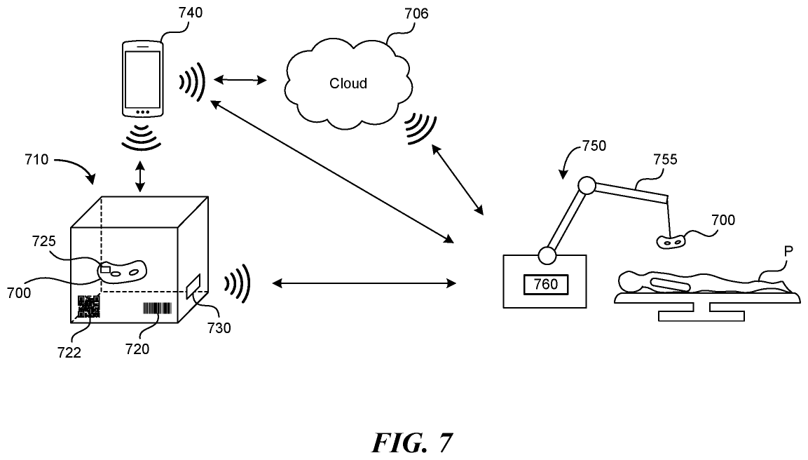

FIG. 7 illustrates various aspects of a procedure for accessing a patient-specific surgical plan (hereinafter referred to as the “surgical plan”) and implanting a patient-specific artificial implant 700 (hereinafter referred to as the “implant 700”) into a patient P using a robotic surgical platform 750 (hereinafter referred to as the “platform 750”). The implant 700 can be any implant previously described herein, such as screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like.

The platform 750 can be configured to perform or otherwise assist with one or more aspects of the operative procedure, including, for example, preparing tissue for an incision, making an incision, making a resection, removing tissue, manipulating tissue, performing a corrective maneuver, delivering the implant to a target site, deploying the implant at the target site, adjusting the implant at the target site, manipulating the implant once it is implanted, securing the implant at the target site, explanting the implant, suturing tissue, etc. For example, the platform 750 can include one or more arms 755 and end effectors for holding various surgical tools (e.g., graspers, clips, needles, needle drivers, irrigation tools, suction tools, staplers, etc.), imaging instruments (e.g., cameras, sensors, etc.), and/or medical devices (e.g., the implant 700) and that enable the platform 750 to perform the one or more aspects of the surgical plan. Although shown as having one arm 755, one skilled in the art will appreciate that the platform 750 can have a plurality of arms (e.g., two, three, four, or more) and any number of joints, linkages, motors, and degrees of freedom. In some embodiments, the platform 750 may have a first arm dedicated to holding one or more imaging instruments, while the remainder of the arms hold various surgical tools. In some embodiments, the tools can be releasably secured to the arms such that they can be selectively interchanged before, during, or after an operative procedure. The arms can be moveable through a variety of ranges of motion (e.g., degrees of freedom) to provide adequate dexterity for performing various aspects of the operative procedure.

The platform 750 can include a control module 760 for controlling operation of the arm(s) 755. In some embodiments, the control module 760 includes a user input device (not shown) for controlling operation of the arm(s) 755. The user input device can be a joystick, a mouse, a keyboard, a touchscreen, an infrared sensor, a touchpad, a wearable input device, a camera- or image-based input device, a microphone, or other user input devices. A user (e.g., a surgeon) can interact with the user input device to control movement of the arm(s) 755. In some embodiments, the control module 760 includes one or more processors for executing machine-readable instructions that, when executed, automatically control operation of the arm 755. In such embodiments, the control module 760 may receive machine-readable instructions specifying one or more steps of a surgical procedure that, when executed by the control module 760, cause the platform 750 to perform the one or more steps of the surgical procedure. For example, the machine-readable instructions may direct the surgical platform 750 to prepare tissue for an incision, make an incision, make a resection, remove tissue, manipulate tissue, perform a corrective maneuver, deliver the implant 700 to a target site, deploy the implant 700 at the target site, adjust a configuration of the implant 700 at the target site, manipulate the implant 700 once it is implanted, secure the implant 700 at the target site, explant the implant 700, suture tissue, and the like. The instructions may therefor include particular instructions for articulating the arm 755 to perform or otherwise aid in the delivery of the patient-specific implant.

The platform 750 can include additional components not expressly shown in FIG. 6. For example, in various embodiments the platform 750 may include one or more displays (e.g., LCD display screen, an LED display screen, a projected, holographic, or augmented reality display (e.g., a heads-up display device or a head-mounted device)), one or more I/O devices (e.g., a network card, video card, audio card, USB, firewire or other external device, camera, printer, speakers, CD-ROM drive, DVD drive, disk drive, or Blu-Ray device), one or more communication devices (e.g., components having VLC, WiMAX, LTE, WLAN, IR communication, PSTN, Radio waves, Bluetooth, and/or Wi-Fi operability) and/or a memory (e.g., random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth). In some embodiments, the foregoing components can be generally similar to the like components described in detail with respect to computing device 200 in FIG. 2.

The implant 700 can be transported to the operating room in packaging 710. As previously described, the surgical plan for implanting the implant 700 into the patient P can be linked to the packaging 710 and/or the implant 700 itself. The surgical plan can be stored locally, remotely, or both locally and remotely. For example, the surgical plan can be stored remotely in a cloud 706, locally in a data storage element 730, or both in the cloud 706 and the data storage element 730. In embodiments in which the surgical plan is stored remotely, the surgical plan may, in addition to or in lieu of being stored in the cloud 706, be remotely stored on a server (e.g., the server 106 shown in FIG. 1) or another networked device or system (e.g., system 100 of FIG. 1). The cloud 706 (or server or other networked data storage system) can receive a request for a particular surgical plan from a computing device, (such as computing device 740 or the computing device 102 shown in FIG. 1) and send the plan to the platform 750. If the surgical plan includes executable instructions, the platform 750 can execute instructions to perform at least a portion of the surgical procedure. In some embodiments, the platform 750 can generate executable instructions based on the surgical plan. For example, the surgical plan can include information about the delivery path, tools, and implantation site. The platform 750 can analyze the surgical plan and develop executable instructions for performing the patient-specific procedure based on the capabilities (e.g., configuration and number of robotic arms, functionality of and effectors, guidance systems, visualization systems, etc.) of the robotic system. This enables the system 100 to be compatible with a wide range of different types of robotic surgery systems.

As previously described, the implant 700 and/or the packaging 710 includes one or more features for accessing, downloading, and/or transmitting the surgical plan and/or other information included within the patient-specific data set. For example, in some embodiments in which the surgical plan is stored remotely, the packaging 710 can include one or more retrieval features, such as a QR code 722 and/or bar code 720. The packaging 710 may include other retrieval features in addition to, or in lieu of, the QR code 722 and the bar code 720, such as any of the retrieval features previously described herein. The QR code 722 and/or the bar code 720 can be used to download or otherwise access the surgical plan. For example, a user can scan the QR code 722 and/or the bar code 720 using a scanner or other computing device 740 (e.g., using a camera on a smart phone) to obtain a unique identifier associated with the implant 700 and the surgical plan. The scanner or other computing device 740 can send (e.g., automatically send) a request including the unique identifier to the cloud 706 to identify the surgical plan associated with the implant 700. The computing device 740 can be in communication with the cloud 706 (or other server) and/or the platform 750 via a wired or wireless connection. In some embodiments, the computing device 740 can be part of the platform 750.

Once identified, the cloud 706 can transmit the surgical plan directly to the platform 750 for execution. In some embodiments, the cloud 706 can transmit the surgical plan to one or more intermediate networked devices, rather than transmitting the surgical plan directly to the platform 750. For example, the cloud 706 may transmit the surgical plan to the computing device 740 and/or the computing device 102, described previously with respect to FIG. 1. A user can review the surgical plan using the computing device before transmitting the surgical plan to the platform 750 for execution. Although shown as being positioned on the packaging 710, the retrieval features may also be positioned on the implant 700 itself, as previously described herein.

In some embodiments, the implant 700 includes one or more patient-specific markings 725. As previously described with respect to the implant 500 shown in FIG. 5, the patient-specific markings 725 can include textual information (e.g., patient name, patient MRN, target implant location, etc.) about the implant 500 and/or the patient P. In some embodiments, the information (e.g., the patient name or patient MRN) can be used to access the surgical plan by inputting (e.g., manually inputting such as typing) the information into the computing device 740. The surgical plan can then be accessed in a manner generally similar to that described previously with respect to the retrieval features 720, 722.

The surgical plan can also be stored locally on a data storage element 730. The data storage element 730 can be any suitable data storage element previously described herein. The data storage element 730 can include a transmitter (not shown) for wirelessly sending the surgical plan to the platform 750 for execution. In some embodiments, the data storage element 730 may send the surgical plan to one or more intermediate devices, rather than sending the surgical plan directly to the platform 750. For example, the data storage element 730 may transmit the surgical plan to the computing device 740 or the computing device 102 (e.g., for surgeon review). The data storage element 730 can also be directly coupled to the computing device 102, the computing device 740, and/or the platform 750 to deliver the surgical plan via a wired or other physical connection. For example, the data storage element 730 can be directly coupled to the computing device 740, and the computing device 740 can be used to transmit the surgical plan to the platform 750. The data storage element may also be incorporated into the implant 700 itself, as previously described herein.

Without being bound by theory, using a robotic surgical platform to perform various aspects of the surgical plans described herein is expected to provide several advantages over conventional operative techniques. For example, use of robotic surgical platforms may improve surgical outcomes and/or shorten recovery times by, for example, decreasing incision size, decreasing blood loss, decreasing a length of time of the operative procedure, increasing the accuracy and precision of the surgery (e.g., the placement of the implant at the target location), and the like. The platform 750 can also avoid or reduce user input errors, e.g., by including one or more scanners for obtaining information from instruments (e.g., instruments with retrieval features), tools, the patient specific implant 700 (e.g., after the implant 700 has been gripped by the arm 755), etc. The platform 750 can confirm use of proper instruments prior and during the surgical procedure. If the platform 750 identifies an incorrect instrument or tool, an alert can be sent to a user that another instrument or tool should be installed. The user can scan the new instrument to confirm that the instrument is appropriate for the surgical plan. In some embodiments, the surgical plan includes instructions for use, a list of instruments, instrument specifications, replacement instruments, and the like. The platform 750 can perform pre- and post-surgical checking routines based on information from the scanners.

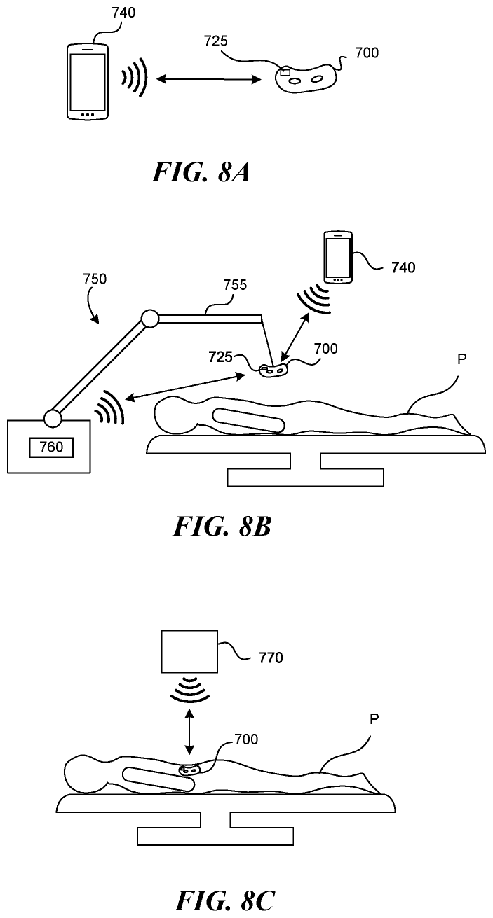

FIGS. 8A-8C illustrate additional representative examples for accessing patient-specific data using the various features described herein before, during, and after a surgical procedure. For simplicity, the implant 700 is shown in FIGS. 8A-8C as only including the patient-specific markings 725. However, although each of FIGS. 8A-8C only illustrate obtaining data using the patient-specific markings 725, one skilled in the art will appreciate that the same or similar steps can be performed if obtaining information by way of the retrieval features 720, 722 (FIG. 7) and/or the data storage element 730 (FIG. 7).

Referring first to FIG. 8A, a user can determine patient-specific information using the computing device 740 to scan, read, image, or otherwise interact with the patient-specific marking 725. For example, in embodiments in which the patient-specific markings 725 include a QR code, a user can scan the QR code using a camera on the computing device 740. In response, the computing device 740 can display the information encoded in the QR code. Of course, in some embodiments the computing device 740 can be used to scan the retrieval feature 720, 722 (shown in FIG. 7) in a generally similar manner to access to a patient-data set stored on a remote server. In some embodiments, the implant 700 is removed from a patient and then read to obtain information used to, for example, select a replacement implant.

Referring next to FIG. 8B, a user can determine patient-specific information using the computing device 740 during a surgical procedure. For example, a user can user the computing device 740 to scan, read, image, or otherwise interact with the patient-specific marking 725 in a manner generally similar to that described with respect to FIG. 8A. Additionally, the surgical platform 750 can be configured to read, or otherwise determine information from, the patient-specific markings 725 (and/or the retrieval features 720, 722 and the data storage element 730) during the surgical procedure. For example, in embodiments in which the patient-specific markings 725 include an alphanumeric code and/or one or more words and/or letters, the robotic surgery platform 750 can include one or more cameras capable of performing optical character recognition (OCR) techniques. In embodiments in which the patient-specific markings 725 include an MR code, a bar code, and/or a QR code, the robotic surgery platform 750 can include one or more cameras capable of scanning, reading, and/or otherwise obtaining information MR codes, bar codes, and/or QR codes.

Referring next to FIG. 8C, a user can determine patient-specific information using after the implant 700 has been implanted in the patient P. For example, an imaging system 770 (e.g., a system for taking one or more of MRI, CAT scan, PET scan, X-Ray, ultrasound, etc.) can be positioned over the portion of the patient P including the implant 700. The imaging system 770 can obtain images of the implant 700, including images of the patient specific markings 725. A computing module (e.g., the computing device 740) can analyze the image data obtained by the imaging system 770 to extract (e.g., via OCR, convoluted neural networks, etc.) information (e.g., the patient-specific marking 725, a retrieval feature such as a bar code, etc.) from the implant 700. The computing module can then construct a virtual model of the reconstructed information and display the virtual model to a user. For example, in embodiments in which the patient-specific marking 725 is text, the computing module can reconstruct a virtual rendering of the text and display the text to a user. In embodiments in which the implant includes a bar code or QR code, the computing module can reconstruct a virtual rendering of the bar code or QR code and display it to the user, such that the user can scan the bar code or QR code to access the corresponding patient-specific data set. In some embodiments, the computing module may perform additional analysis or transformation while reconstructing the information. For example, in some embodiments the patient-specific markings 725 may include machine-readable information (e.g., a bar-code, an alphanumeric code, shapes, etc.), and the computing module may extract the machine-readable information and transform (e.g., decode) the machine-readable information into human-readable information rendered virtually and displayed to the user.

### CONCLUSION

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2018, titled “SYSTEMS AND METHODS FOR ASSISTING AND AUGMENTING SURGICAL PROCEDURES”;
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY”;
U.S. application Ser. No. 16/207,116, filed on Dec. 1, 2018, titled “SYSTEMS AND METHODS FOR MULTI-PLANAR ORTHOPEDIC ALIGNMENT”;
U.S. application Ser. No. 16/352,699, filed on Mar. 13, 2019, titled “SYSTEMS AND METHODS FOR ORTHOPEDIC IMPLANT FIXATION”;
U.S. application Ser. No. 16/383,215, filed on Apr. 12, 2019, titled “SYSTEMS AND METHODS FOR ORTHOPEDIC IMPLANT FIXATION”;
U.S. application Ser. No. 16/569,494, filed on Sep. 12, 2019, titled “SYSTEMS AND METHODS FOR ORTHOPEDIC IMPLANTS”;
U.S. application Ser. No. 16/699,447, filed on Nov. 29, 2019, titled “SYSTEMS AND METHODS FOR ORTHOPEDIC IMPLANTS”;
U.S. application Ser. No. 17/085,564, filed on Oct. 30, 2020, titled “SYSTEMS AND METHODS FOR DESIGNING ORTHOPEDIC IMPLANTS BASED ON TISSUE CHARACTERISTICS”;
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS”;
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS”;
U.S. application Ser. No. 17/100,396, filed Nov. 20, 2020, titled “PATIENT-SPECIFIC VERTEBRAL IMPLANTS WITH POSITIONING FEATURES”; and
U.S. application Ser. No. 17/342,439, filed Jun. 8, 2021, titled “PATIENT-SPECIFIC MEDICAL PROCEDURES AND DEVICES, AND ASSOCIATED SYSTEMS AND METHODS.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

1. A system for providing medical care, the system comprising:
a patient-specific implant designed to be implanted at a target site within a particular patient, wherein the patient-specific implant includes at least one structural feature designed to engage one or more identified anatomical structures at the target site;
a patient-specific surgical plan including instructions for implanting the patient-specific implant at the target site such that the at least one structural feature engages the one or more identified anatomical structures;
a data storage module having a memory storing the patient-specific surgical plan; and
a retrieval module configured to transmit the patient-specific surgical plan from the data storage module to a surgical platform configured to execute one or more aspects of the patient-specific surgical plan.

2. The system of claim 1 wherein the data storage module includes a data storage element positioned on or within (i) the patient-specific implant or (ii) packaging configured to hold the patient-specific implant, and wherein the retrieval module includes one or more transmitters for transmitting the patient-specific surgical plan from the data storage element to the surgical platform.

3. The system of claim 2 wherein the one or more transmitters include an antenna, a proximity sensor, and/or a location sensor.

4. The system of claim 1 wherein the data storage module includes a cloud-based storage module or a remote server, and wherein the retrieval module includes one or more retrieval features for downloading the patient-specific surgical plan stored on the cloud-based storage module or the remote server.

5. The system of claim 4 wherein the one or more retrieval features include an MR code, a QR code, and/or a bar code.

6. The system of claim 1 wherein the retrieval module is positioned on or within the patient-specific implant.

7. The system of claim 1, further comprising packaging for the patient specific implant, wherein the retrieval feature is positioned on or within the packaging.

8. The system of claim 1 wherein the surgical platform is a robotic surgical platform, and wherein the instructions include machine-readable instructions that, when executed by the robotic surgical platform, cause the robotic surgical platform to perform one or more aspects of the patient-specific surgical plan.

9. The system of claim 8 wherein the one or more aspects include preparing tissue for an incision, making an incision, performing a resection, removing tissue, manipulating tissue, performing a corrective maneuver, delivering the patient-specific implant to the target site, deploying the patient-specific implant at the target site, adjusting a configuration of the patient-specific implant at the target site, manipulating the patient-specific implant after it is implanted at the target site, securing the patient-specific implant at the target site, and/or suturing tissue.

10. The patient-specific implant of claim 1 wherein the instructions include computer-readable instructions that, when executed by a processor coupled to a display, cause at least one step for performing the patient-specific surgical plan to be displayed via the display.

11. A patient-specific implant designed to be implanted at a target position within a particular patient, the patient-specific implant comprising:
an implant body configured to interface with one or more identified anatomical structures at and/or proximate the target position; and
a data storage element positioned on and/or within the implant body and configured to be implanted with the patient-specific implant, wherein the data storage element has memory storing data, wherein the data includes (i) first data specifying at least one step of a patient-specific surgical plan for implanting the patient-specific implant at the target position, and (ii) second data specifying at least one characteristic of the patient-specific implant.

12. The patient-specific implant of claim 11 wherein
the implant body includes at least one structural feature designed to engage a respective one of the one or more identified anatomical structures, and
the first data includes instructions for performing the patient-specific surgical plan to deliver the implant body to the target position such that the at least one structural feature engages the respective one of the one or more identified anatomical structures.

13. The patient-specific implant of claim 12 wherein the instructions include machine-readable instructions that, when executed by a robotic surgical platform, cause the robotic surgical platform to perform one or more aspects of the patient-specific surgical plan.

14. The patient-specific implant of claim 12 wherein the instructions include computer-readable instructions that, when executed by a processor coupled to a display, cause at least one step for performing the patient-specific surgical plan to be displayed via the display.

15. The patient-specific implant of claim 12 wherein the instructions are encoded, and wherein the instructions are configured to be decoded and used for performing the patient-specific surgical plan.

16. The patient-specific implant of claim 11 wherein the data includes the target position.

17. The patient-specific implant of claim 11 wherein the data includes one or more of a pre-operative plan, a surgical procedure, a surgical approach, and/or one or more surgical steps.

18. The patient-specific implant of claim 11 wherein the second data includes one or more of implant dimensions, implant composition, unique implant identification code, implant part number, implant lot code, implant manufacture date, implant implantation date, implant expiration date, implant sterilization history, and/or implant manufacturing history.

19. The patient-specific implant of claim 11 wherein the data storage element further includes third data associated with the particular patient.

20. The patient-specific implant of claim 19 wherein the third data includes patient information, patient medical history, a patient medical record number, and/or physician notes about the patient.

21. The patient-specific implant of claim 11 wherein the data is accessible after the patient-specific implant is implanted in the patient.

22. The patient-specific implant of claim 11, further comprising a transmitter that, when the patient-specific implant is implanted in the patient, is configured to transmit at least a portion of the first data and/or the second data from the data storage element to a device positioned external to the patient.

23. The patient-specific implant of claim 22 wherein the transmitter is configured to transmit the portion of the first data and/or the second data to the external device when the transmitter is exposed to an electric and/or magnetic field.

24. The patient-specific implant of claim 22 wherein the implant body includes a structural feature that acts as the transmitter.

25. The patient-specific implant of claim 24 wherein the transmitter includes an antenna.

26. The patient-specific implant of claim 11 wherein the patient-specific implant includes a position sensor that, when the patient-specific implant is implanted in the patient, is configured to wirelessly transmit information about the location of the patient-specific implant.

27. The patient-specific implant of claim 26 wherein the position sensor is included on and/or in the data storage element.

28. The patient-specific implant of claim 11 wherein the data storage element is an implantable microchip.

29-91. (canceled)
